(19) United States
(12) Patent Application Publication
(10) Pub. No.: US 2016/0279368 A1
(43) Pub. Date: Sep. 29, 2016

(54) Nasal Mask for Ventilation of Patient

(71) Applicant: Derek Isenberg, Haddon Heights, NJ (US)

(72) Inventor: Derek Isenberg, Haddon Heights, NJ (US)

(21) Appl. No.: 14/884,982

(22) Filed: Oct. 16, 2015

Related U.S. Application Data
(60) Provisional application No. 62/137,259, filed on Mar. 24, 2015.

(51) Int. Cl. A61M 16/06; A61M 16/08

(52) U.S. Cl. CPC A61M 16/0605; A61M 16/0816; A61M 16/0683; A61M 2202/0208; A61M 2016/0661; A61M 2205/02

(57) ABSTRACT

A nasal mask for ventilating a patient is disclosed. The mask for delivering oxygen to a patient comprises a shaped wall defining a mask body and terminating in an edge, a buffer disposed on the edge, and a cylindrical spout disposed from the mask body. The shaped wall has a narrow first end, a wider second end disposed opposite the narrow first end, and a central axis extending longitudinally along the mask body. The buffer defines a plane. The cylindrical spout is disposed at an angle with the plane. The cylindrical spout is disposed toward the wider second end and is in line with the central axis. The angle being between sixty degrees and eighty degrees to the plane. The mask may further comprise a connection means and a strap connected to the connection means.

### PRIORITY

This application claims priority to U.S. Provisional Patent Application Ser. No. 62/137,259 filed Mar. 24, 2015.

### TECHNICAL FIELD

This invention pertains generally to medical devices and more particularly to a shaped nasal mask for ventilation of a patient which creates an airtight seal.

### BACKGROUND OF THE INVENTION

Patients of doctors or hospitals may encounter breathing problems. These situations may often arise when there is a serious injury or physical event which is experienced by the patient. For instance, if a patient experiences a stroke or heart attack and loses consciousness, then the patient may be unable to breath during that time and for a short time after. Emergency medical personnel are trained to assist the patient with breathing. Medical personnel, such as nurses or EMTs, can assist the patient with breathing by providing CPR or via a bag valve mask.

Bag valve masks are utilized to provide positive pressure ventilation to patients and may be used to resuscitate a patient. Current masks which are utilized have a face mask portion to cover the mouth and nose of a patient. Current masks are provided in a series of shapes. The most common shape is a teardrop shape with a smaller width at the top and a larger width at the bottom of the mask. The smaller area is configured to fit over a patient's nose with the wider area is configured to fit over a patient's mouth. The outer perimeter of the mask has a soft buffer area, allowing the mask to be pressed onto the patient's face. These masks do not create a perfect seal with the patient's airway. The shape of individual patient's faces can vary depending on genetic bone structure or if a patient is missing any or all of his teeth. Also, some patients may have an extraordinary amount of facial hair which impedes the placement of a face mask over the patient's mouth and nose. In these instances, gaps may be present around the perimeter of the face mask on the patient. These gaps permit the leakage of air and create inefficiencies in the airflow when a patient is being ventilated. What is needed is a mask for use on a patient which maintains an air seal between the mask and the patient's face.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The invention is directed toward a mask for delivering oxygen to a patient comprising a shaped wall defining a mask body and terminating in an edge, a buffer disposed on the edge, and a cylindrical spout disposed from the mask body. The shaped wall has a narrow first end, a wider second end disposed opposite the narrow first end, and a central axis extending longitudinally along the mask body. The buffer defines a plane. The cylindrical spout is disposed at an angle with the plane. The cylindrical spout is disposed toward the wider second end and is in line with the central axis. The angle being between sixty degrees and eighty degrees to the plane.

In another embodiment the mask further comprises a flat surface being disposed between the spout and the shaped wall and being substantially parallel to the plane. In another embodiment the angle is between sixty-five degrees and seventy-five degrees. In another embodiment the angle is substantially seventy degrees. The mask may be composed of thermoplastic. In another embodiment the mask further comprises a connection means disposed on the shaped wall and a strap connected to the connection means.

Still other embodiments of the present invention will become readily apparent to those skilled in this art from the following description wherein there is shown and described the embodiments of this invention, simply by way of illustration of the best modes suited to carry out the invention. As it will be realized, the invention is capable of other different embodiments and its several details are capable of modifications in various obvious aspects all without departing from the scope of the invention. Accordingly, the drawing and descriptions will be regarded as illustrative in nature and not as restrictive.

### BRIEF DESCRIPTION OF THE DRAWINGS

FIG. 1 is a front view of the nasal mask;

FIG. 2 is a back view of the nasal mask;

FIG. 3 is a side view of the nasal mask;

FIG. 4 is a cut away side view of the nasal mask;

FIG. 5 is a perspective view of the nasal mask on a patient;

FIG. 6 is a side view of an alternative embodiment of the nasal mask;

FIG. 7 is perspective view of an alternative embodiment of the nasal mask; and

FIG. 8 is a bottom view of the alternative embodiment of the nasal mask.

### DESCRIPTION OF THE INVENTION

The claimed subject matter is now described with reference to the drawings. In the following description, for purposes of explanation, numerous specific details are set forth in order to provide a thorough understanding of the claimed subject matter. It may be evident, however, that the claimed subject matter may be practiced with or without any combination of these specific details, without departing from the spirit and scope of this invention and the claims.

The invention is directed toward a nasal mask to be used for ventilation of a patient. The nasal mask creates an airtight seal around the nose of the patient to avoid loss of air pressure and ensure full ventilation of the patient. Referring to FIG. 1, the preferred embodiment is displayed. FIG. 1 illustrates a front view of the mask 10. The nasal mask comprises a mask 10 and a connection means 20. The mask 10 has a central opening 12. The central opening 12 is configured to provide a pathway for airflow during ventilation. The central opening 12 is configured to provide an attachment point for a bag valve or breathing machine. The connection means 20 provides a point of attachment for removably securing the mask 10 to the patient's face. The connection means 20 may be any form or structure permitting an attachment of a tie, a clip, a band, or any other type of component which preferably wraps around the head of the patient to maintain a seal between the mask 10 and the patient's face.

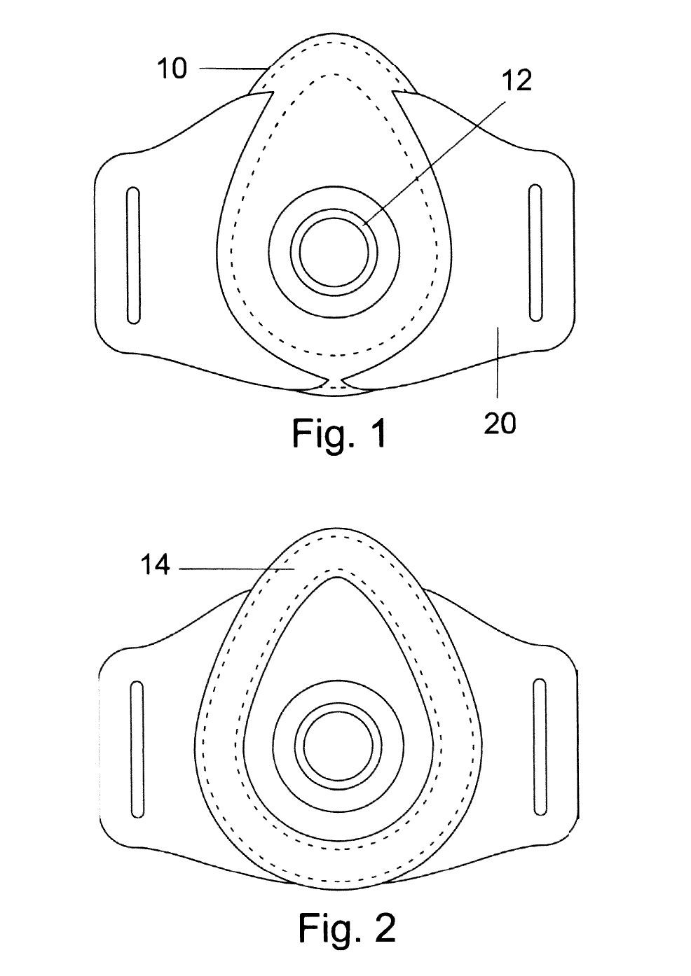

Referring to FIG. 2, a back view of the mask 10 is displayed. The back side of the mask comprises a buffer 14. The buffer 14 surrounds the outer periphery of the mask 10. The buffer 14 provides a compressible cushion form to provide a more comfortable compression against the skin of the patient. The buffer 14 ensures an airtight seal is formed around the nose of the patient.

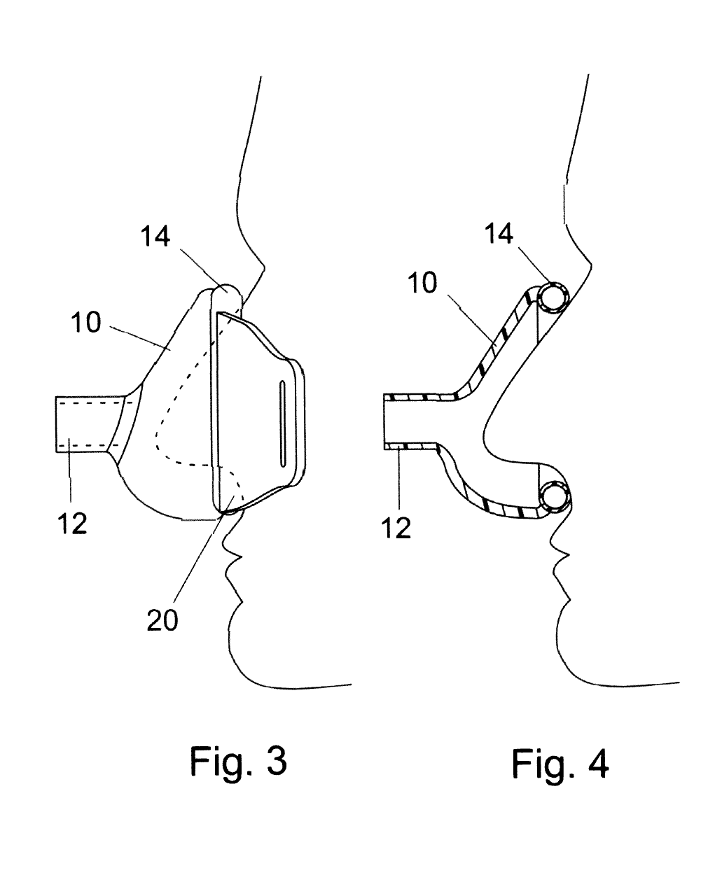

Referring to FIG. 3 and FIG. 4, a side view of the mask 10 is illustrated being worn by a patient. The mask 10 has an internal cavity shaped for receiving the patient's nose. The connection means 20 lies on each side the patient's nose. The buffer 14 surrounds the entire periphery of the patient's nose to ensure an airtight seal is formed. The central opening 12 provides the opening for airflow from a bag valve or breathing assistance machine or apparatus.

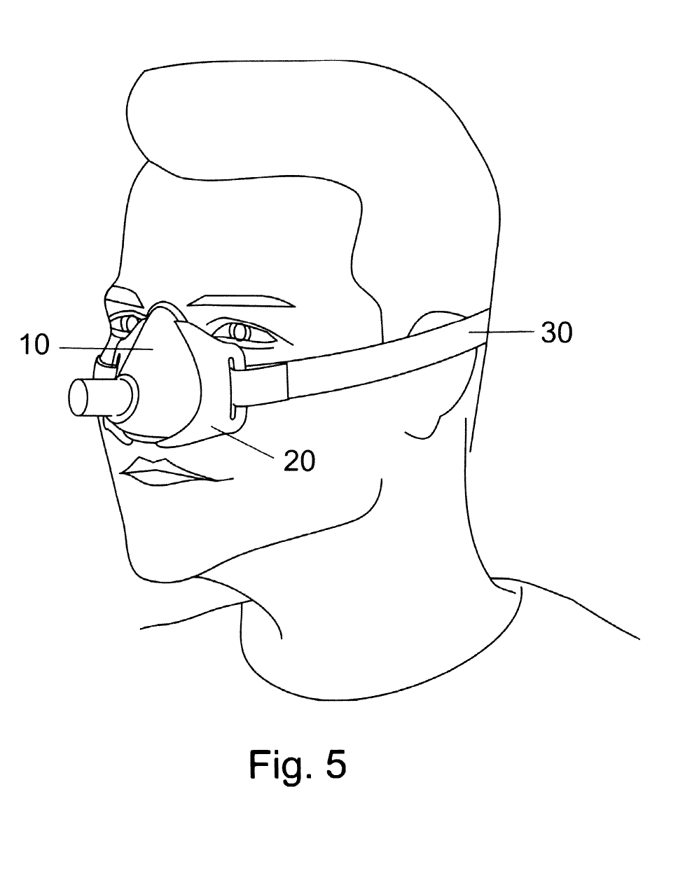

Referring to FIG. 5, a mask 10 is illustrated being worn on a patient. The connection means 20 is connected to the mask and is connected to a strap 30. The strap 30 extends around the patient's head and holds the mask 10 in place on the patient's face.

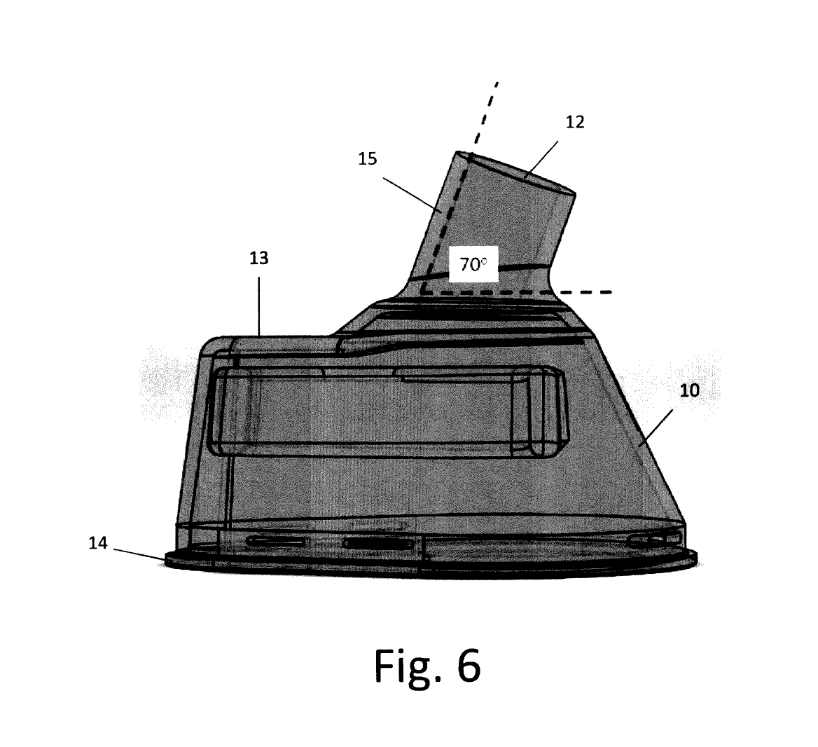

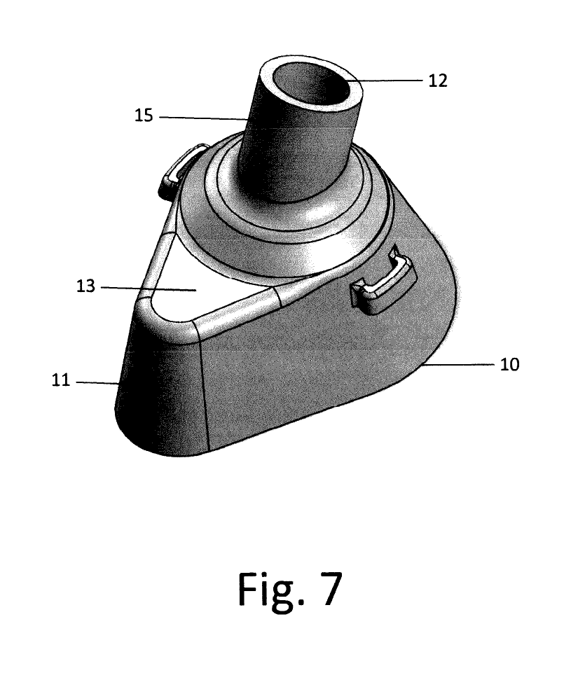

Referring to FIG. 6 through FIG. 8, an alternative embodiment of the mask 10 is displayed. In this embodiment the mask 10 has a flat surface 13 disposed on the opposite side of the mask 10 from the buffer 14. The flat surface 13 is connected on all sides by a shaped wall 11. In the preferred embodiment, the shaped wall 11 is substantially triangular in shape and is disposed between the flat surface 13 and the buffer 14. The flat surface 13 is substantially planar. The plane of the flat surface 13 is substantially parallel to the plane formed by the edge of the buffer 14. Disposed from the flat surface 13 of the mask 10 is a spout 15. The spout 15 terminates in the central opening 12. The spout 15 is substantially cylindrical in shape. The spout 15 has a central axis. The central axis of the spout 15 is disposed at an oblique angle to the plane of the flat surface 15. The central axis of the spout 15 may be at any angle to the plane of the flat surface 15. In the preferred embodiment the central axis of the spout 15 is disposed at a seventy degree angle to the flat surface 13 of the mask 10. The central axis of the spout 15 may be disposed within a range of angles, such as sixty degrees to eighty degrees. The angle of the spout 15 to the flat surface 13 provides an increased efficiency in the operation of the mask 10. At the oblique angle, air entering the central opening 12 passes through the spout 15 and is directed in an upward flow to flow up the nostrils of the patient. In this configuration, the mask 10 operates at an increased efficiency—increasing the flow of air into the nasopharyngeal cavity of the patient and into the lungs, thereby increasing the uptake of oxygen by the patient.

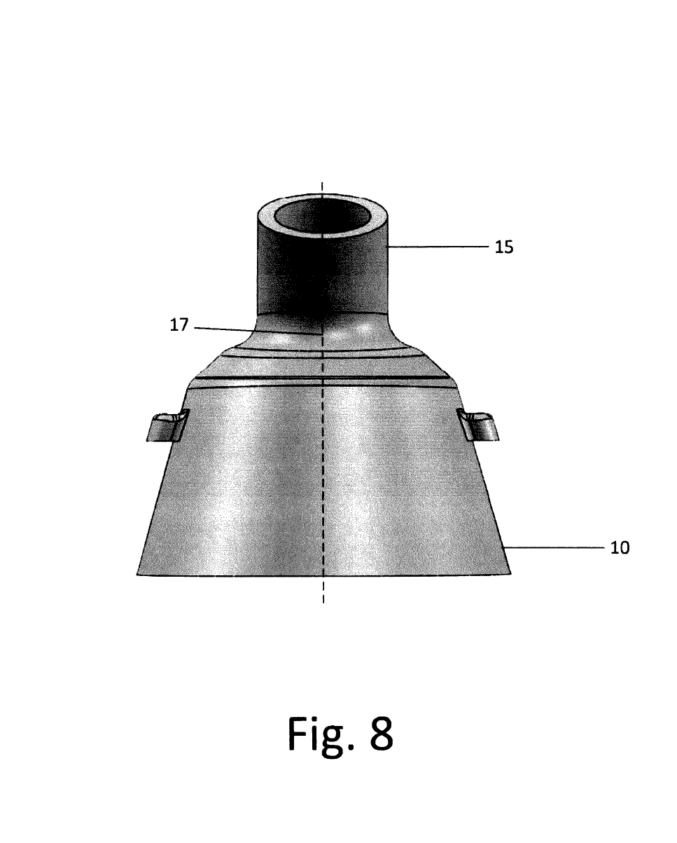

As shown in FIG. 8, the mask 10 has a central axis 17 running along the length of the mask 10. The central axis 17 divides the mask 10 into two mirrored sides. The spout 15 is disposed in line with the central axis 17.

The mask 10 may be made of any type of flexible material. Preferably the mask 10 is made of silicone or some other flexible elastomer material. Optionally the buffer 14 may be composed of a silicone or flexible elastomer while the other components are composed of a different polymer material. The connection means 20 may be made of any type of material. In the preferred embodiment, the connection means 20 is composed of the same material as the mask 10. The strap 30 may be made of any type of material but preferably is a stretchable material such as a fabric containing spandex fibers, an elastomer, or synthetic rubber. The mask 10 can be of any size but preferably is no larger than necessary to fully encircle the nose of the patient. The central opening 12 may be of any size. In the preferred embodiment the central opening 12 is 16 mm in diameter.

The invention is also directed toward a method of ventilating a patient utilizing a nasal mask 10. The method comprises placing a nasal mask 10 over the nose of a patient. The user presses the nasal mask 10 over the nose of the patient and presses the buffer 14 of the nasal mask 10 against the skin of the patient to ensure a proper airtight seal. The buffer 14 surrounds the nose of the patient and creates an airtight seal. The user then connects a bag valve or breathing machine to the central opening 12 for ventilating the patient. The user then can connect a connecting strap 30 to a connection means 20 on the mask 10. The user can position the connecting strap 30 behind the head of the patient to ensure the mask 10 is fit secure on the patient's face. When ventilating the patient, the airflow flows through the mask 10 and into the nasopharyngeal cavity of the patient. The positive pressure pushes the air through the nasopharyngeal cavity, pushing the uvula into an open position. The air can then flow into the patient's lungs.

The invention may be utilized as a stand-alone device or be utilized with other components. For instance, the mask 10 may be connected to a silicone tube. The silicone tube is then strung through the nasopharyngeal cavity of the patient, through the patient's uvula, and into the patient's esophagus.

The invention may be realized with any number of additional structures or variations in the mask 10. For instance, the mask 10 may have additional internal air valves or seals situated within the mask 10, or near the central opening 12, to prevent air leakage and loss of pressure.

What has been described above includes examples of the claimed subject matter. It is, of course, not possible to describe every conceivable combination of components or methodologies for purposes of describing the claimed subject matter, but one of ordinary skill in the art can recognize that many further combinations and permutations of such matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

1) A mask for delivering oxygen to a patient comprising
a) a shaped wall defining a mask body and terminating in an edge, said shaped wall having a narrow first end, a wider second end disposed opposite said narrow first end, and a central axis extending longitudinally along the mask body;
i) a buffer disposed on said edge, said buffer defining a plane;
b) a cylindrical spout disposed from said mask body
i) wherein said cylindrical spout is disposed at an angle with said plane with said cylindrical spout being disposed toward said wider second end being in line with said central axis;
ii) said angle being between sixty degrees and eighty degrees to said plane.

2) The mask as in claim 1 further comprising a flat surface being disposed between said spout and said shaped wall and being substantially parallel to said plane.

3) The mask as in claim 1 wherein said angle is between sixty-five degrees and seventy-five degrees.

4) The mask as in claim 1 wherein said angle is substantially seventy degrees.

5) The mask as in claim 1 wherein said mask is composed of a thermoplastic.

6) The mask as in claim 2 wherein said angle is between sixty-five degrees and seventy-five degrees.

7) The mask as in claim 2 wherein said angle is substantially seventy degrees.

8) The mask as in claim 2 wherein said mask is composed of a thermoplastic.

9) The mask as in claim 7 wherein said mask is composed of a thermoplastic.

10) The mask as in claim 1 further comprising a connection means disposed on said shaped wall and a strap connected to said connection means.

11) The mask as in claim 2 further comprising a connection means disposed on said shaped wall and a strap connected to said connection means.

12) The mask as in claim 4 further comprising a connection means disposed on said shaped wall and a strap connected to said connection means.

13) The mask as in claim 6 further comprising a connection means disposed on said shaped wall and a strap connected to said connection means.

14) The mask as in claim 7 further comprising a connection means disposed on said shaped wall and a strap connected to said connection means.

15) The mask as in claim 8 further comprising a connection means disposed on said shaped wall and a strap connected to said connection means.

16) The mask as in claim 9 further comprising a connection means disposed on said shaped wall and a strap connected to said connection means.
